(19) United States
(12) Patent Application Publication
(10) Pub. No.: US 2026/0055357 A1
(43) Pub. Date: Feb. 26, 2026

(54) ANOMALY DETECTION APPARATUS, METHOD, AND PROGRAM

(71) Applicant: NTT, Inc, Tokyo (JP)

(72) Inventors: Nobuaki ENDO, Musashino-shi, Tokyo (JP); Kazuhiro TAKAYA, Musashino-shi, Tokyo (JP)

(21) Appl. No.: 19/105,892

(22) PCT Filed: Sep. 22, 2022
(86) PCT No.: PCT/JP2022/035446

§ 371 Date: Feb. 24, 2025

(51) Int. Cl. C12M 1/34; C12M 1/36; C12M 3/06

(52) U.S. Cl. CPC C12M 41/46; C12M 23/16; C12M 41/48

(57) ABSTRACT

An abnormality detection device according to an embodiment includes a measurement unit that measures a culture state of a sample of a culture target that is a culture target during mass culturing by a mass culturing device and that is fed to a microchannel chip and cultured under a set culture condition, and a detection unit that detects an abnormality in a culture state of a sample of the culture target by comparing an abnormality detection model in which a criterion for determining an abnormality in the culture state indicating that the culture state of the sample of the culture target is different from a normal culture state is defined for each of a plurality of culture conditions, and the culture state measured by the measurement unit for the sample and the set culture condition.

### TECHNICAL FIELD

An embodiment of the present invention relates generally to an abnormality detection device, a method, and a program.

### BACKGROUND ART

Culture targets such as microalgae have been utilized in various applications such as food, environmental purification, and production of biofuels (see, for example, Patent Literature 1 and Non Patent Literature 1). In mass culturing (sometimes referred to as large-scale culture) of microalgae, it is necessary to avoid problems such as that microalgae having desired properties cannot be obtained because contamination organisms are mixed in a culturing device.

### CITATION LIST

### Patent Literature

Patent Literature 1: JP 2014-200211 A

### Non Patent Literature

Non Patent Literature 1: Satoko KOMATSU, Noriyuki TAKEUCHI, Hiroaki FUKUDA, “Development of Outdoor Cultivation Process for Green Microalga Pseudococcomyxasp. KJ Strain”, Journal of the Japan Institute of Energy, Enermix, 96, 56-62 (2017).

### SUMMARY OF INVENTION

### Technical Problem

In the conventional method, culture data of microalgae by a mass culturing device is directly acquired in real time, or an abnormality during mass culturing of microalgae is detected using past culture data. Therefore, at the time of abnormality detection, there is a problem that a large influence has already been exerted on the mass culturing device, for example, an influence of occurrence of contamination to such an extent that it is not possible to be recovered or a decrease in culture efficiency due to genetic mutation, or a cost related to acquisition of culture data is large.

While there is a possibility that contamination occurs in a strain exhibiting excellent characteristics under specific culture conditions or a genetic mutation occurs during the mass culturing, in the conventional method, there is a problem that the time for satisfying specific culture conditions is short in the mass culturing device, a relative proportion of mutant strains does not increase, and the contamination occurrence or the genetic mutation cannot be detected.

The present invention has been made in view of the above circumstances, and an object of the present invention is to provide an abnormality detection device, a method, and a program capable of appropriately detecting an abnormality in a culture state of a culture target during mass culturing.

### Solution to Problem

An abnormality detection device according to an aspect of the present invention includes a measurement unit that measures a culture state of a sample of a culture target that is a culture target during mass culturing by a mass culturing device and that is fed to a microchannel chip and cultured under a set culture condition, and a detection unit that detects an abnormality in a culture state of a sample of the culture target by comparing an abnormality detection model in which a criterion for determining an abnormality in the culture state indicating that the culture state of the sample of the culture target is different from a normal culture state is defined for each of a plurality of culture conditions, and the culture state measured by the measurement unit for the sample and the set culture condition.

An abnormality detection method according to an aspect of the present invention is a method performed by an abnormality detection device, the method including measuring, by a measurement unit of the abnormality detection device, a culture state of a sample of a culture target that is a culture target during mass culturing by a mass culturing device and that is fed to a microchannel chip and cultured under a set culture condition, and detecting, by a detection unit of the abnormality detection device, an abnormality in a culture state of a sample of the culture target by comparing an abnormality detection model in which a criterion for determining an abnormality in the culture state indicating that the culture state of the sample of the culture target is different from a normal culture state is defined for each of a plurality of culture conditions, and the culture state measured by the measurement unit for the sample and the set culture condition.

### Advantageous Effects of Invention

According to the present invention, it is possible to appropriately detect an abnormality in a culture state of a culture target during mass culturing.

### BRIEF DESCRIPTION OF DRAWINGS

FIG. 1 is a diagram illustrating an application example of an abnormality detection system according to an embodiment of the present invention.

FIG. 2 is a diagram illustrating a configuration example of a microchannel system.

FIG. 3 is a block diagram illustrating an example of a functional configuration of a control server.

FIG. 4 is a block diagram illustrating an example of a functional configuration of an abnormality detection server.

FIG. 5 is a flowchart illustrating an example of a procedure of creating an abnormality detection model.

FIG. 6 is a table illustrating an example of an abnormality detection model in a tabular form.

FIG. 7 is a flowchart illustrating an example of procedures of mass culturing and abnormality detection.

FIG. 8 is a table illustrating an example of an abnormality determination result in a tabular form.

FIG. 9 is a view illustrating an example of a notification screen for abnormality detection.

FIG. 10 is a block diagram illustrating an example of a hardware configuration of the abnormality detection Server according to the embodiment of the present invention.

### DESCRIPTION OF EMBODIMENTS

Hereinafter, an embodiment according to the present invention will be described with reference to the drawings.

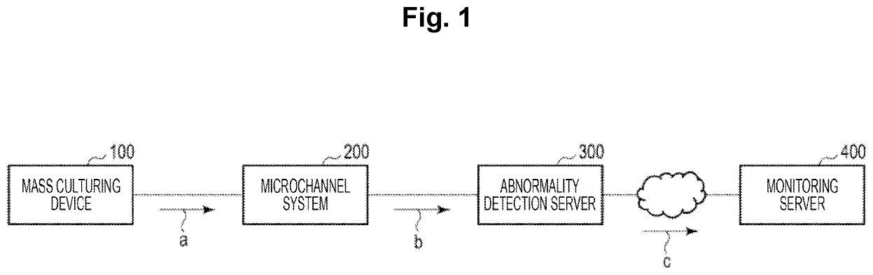

FIG. 1 is a diagram illustrating an application example of an abnormality detection system according to an embodiment of the present invention.

As illustrated in FIG. 1, the abnormality detection system according to the present embodiment includes a mass culturing device 100, a microchannel system 200, an abnormality detection server 300, and a monitoring server 400. The abnormality detection server 300 and the monitoring server 400 can be connected via, for example, a network.

Next, abnormality detection model creation by the abnormality detection system will be described.

In the present embodiment, the abnormality detection system creates an abnormality detection model, which is a model for detecting an abnormality during culture, before starting mass culturing of the culture target. Hereinafter, an application example when the culture target is microalgae will be described, but the embodiment is not limited thereto, and is applicable even when the culture target is, for example, a microorganism, a cell, a bacterium, a yeast, or the like.

First, in the microchannel system 200, a culture target strain to be used as a standard for abnormality determination during culture, that is, an ideal culture strain having no contamination occurrence or genetic mutation is cultured under various culture conditions which are environmental conditions to be considered in culture, and culture data related to the culture is acquired and transmitted to the abnormality detection server 300.

Based on the culture data, the abnormality detection server 300 creates an abnormality detection model.

Next, mass culturing and abnormality detection by the abnormality detection system will be described.

First, mass culturing of a culture strain of microalgae is started by the mass culturing device 100.

The mass culturing device 100 feeds a trace sample of a culture solution containing a culture target strain during mass culturing to the microchannel system 200 (reference sign a in FIG. 1).

In the microchannel system 200, the trace sample is cultured under various culture conditions, and culture data related to the culture is generated and transmitted to the abnormality detection server 300 (reference sign b in FIG. 1).

Based on the culture data and the abnormality detection model created in advance as described above, the abnormality detection server 300 detects an abnormality during mass culturing of microalgae, that is, a change in culture efficiency due to occurrence of contamination, genetic mutation, or the like.

When an abnormality is detected by the abnormality detection server 300, a response at the time of abnormality is performed. Examples of the response at the time of abnormality include (1) notification from the abnormality detection server 300 to the monitoring server 400 (reference sign c in FIG. 1) and (2) transmission of a valve control command for selecting a sample at the time of abnormality detection from the abnormality detection server 300 to a control server 209.

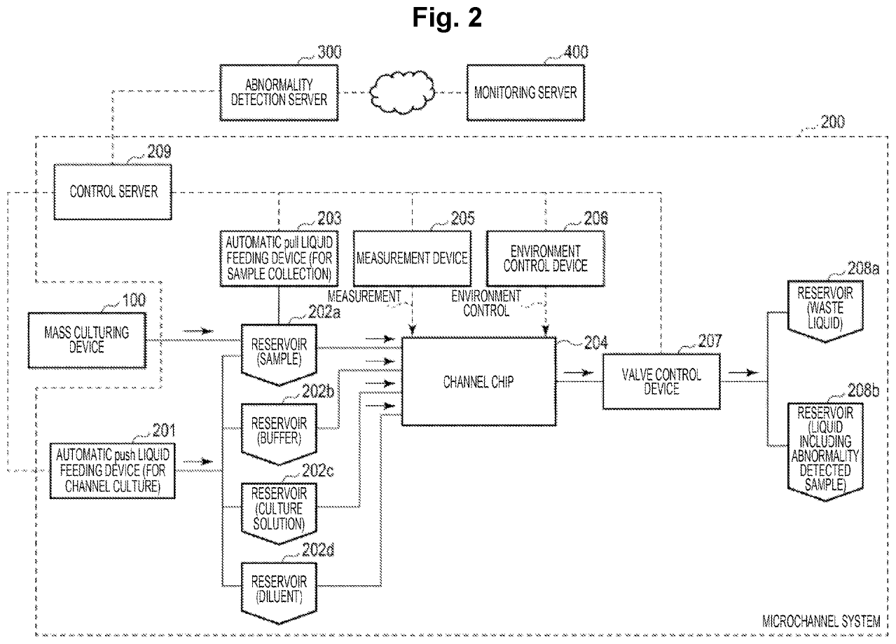

FIG. 2 is a diagram illustrating a configuration example of a microchannel system.

As illustrated in FIG. 2, the microchannel system 200 includes an automatic push liquid feeding device (for channel culture) 201, a reservoir (for sample) 202a, a reservoir (for buffer) 202b, a reservoir (for culture solution) 202c, a reservoir (for diluent) 202d, an automatic pull liquid feeding device (for sample collection) 203, a channel chip (microchannel chip) 204, a measurement device (microscope system or the like) 205, an environment control device (temperature, Illuminance, and the like) 206, a valve control device 207, a reservoir (for waste liquid) 208a, a reservoir (for liquid including sample at the time of abnormality detection) 208b, and a control server 209.

The automatic push liquid feeding device 201 and the automatic pull liquid feeding device 203 may include a pump, a channel regulator, a flow rate sensor, and the like.

As illustrated in FIG. 2, the culture strain of microalgae and the trace sample containing the culture solution from the mass culturing device 100 are fed to the reservoir (sample) 202a. The control server 209 controls liquid feeding from the reservoir (for sample) 202a, the reservoir (for buffer) 202b, the reservoir (for culture solution) 202c, and the reservoir (for diluent) 202d to the channel chip 204 by the automatic push liquid feeding device (for channel culture) 201.

The automatic pull liquid feeding device 203 collects a trace sample before liquid feeding from the reservoir (for sample) 202a to the microchannel system 200 in accordance with a control command from the control Server 209.

The measurement device 205 includes, for example, a microscope system or the like, and can measure the specific growth rate of the trace sample fed to the channel chip 204, that is, the increase amount of the cell amount per unit time a plurality of times in accordance with a control command from the control server 209. The target of this measurement may be, for example, a specific substrate consumption rate, a product specific production rate, or a specific oxygen consumption rate. It is assumed that the measurement results over the plurality of times are distributed according to a normal distribution.

In accordance with a control command from the control server 209, the environment control device 206 controls the temperature of the trace sample flowing through the channel in the channel chip 204, the illuminance of irradiation light to the trace sample, and the like, which are types of culture conditions related to the trace sample flowing through the channel chip 204, via an air conditioning node and a lighting device (not illustrated). In addition, the control server 209 controls liquid feeding from various reservoirs to the microchannel system 200 by the automatic push liquid feeding device 201 in order to control ph (hydrogen ion concentration index), pCO₂ (carbon dioxide partial pressure), nutrient salt concentration, and the like, which are types of culture conditions related to the trace sample flowing through the channel chip 204.

The nutrient salt concentration may include a plurality of kinds of concentrations, for example, concentrations of phosphorus, nitrogen, metal ions, and silicic acid. The metal ion is, for example, an iron ion.

The valve control device 207 switches the liquid feeding destination of the trace sample from the channel chip 204 between the reservoirs 208a and 208b by controlling a valve, not illustrated, provided in the channel between the outlet side of the channel chip 204 and the reservoirs 208a and 208b in accordance with a control command from the control server 209.

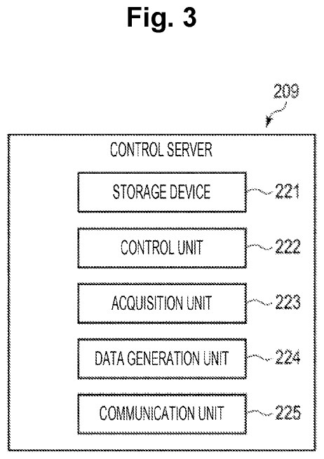

FIG. 3 is a block diagram illustrating an example of a functional configuration of a control server.

As illustrated in FIG. 3, the control server 209 includes a storage device 221, a control unit 222, an acquisition unit 223, a data generation unit 224, and a communication unit 225. The storage device 221 is provided with, for example, a working memory related to processing by the data generation unit 224. Functions and the like of the respective units will be described later.

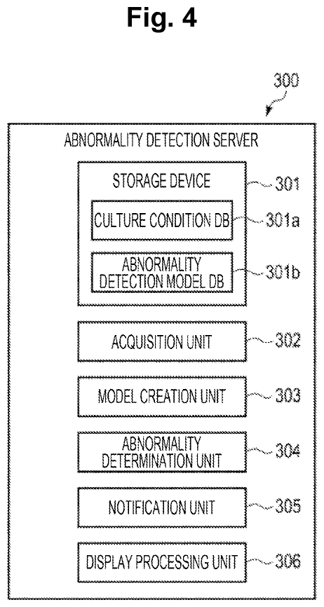

FIG. 4 is a block diagram illustrating an example of a functional configuration of the abnormality detection server.

As illustrated in FIG. 4, the abnormality detection server 300 includes a storage device 301, an acquisition unit 302, a model creation unit 303, an abnormality determination unit 304, a notification unit 305, and a display processing unit 306. The storage device 301 is provided with a culture condition database (DB) 301a and an abnormality detection model DB 301b, and is also provided with, for example, a working memory related to processing by the model creation unit 303 and the abnormality determination unit 304. Functions and the like of the respective units will be described later.

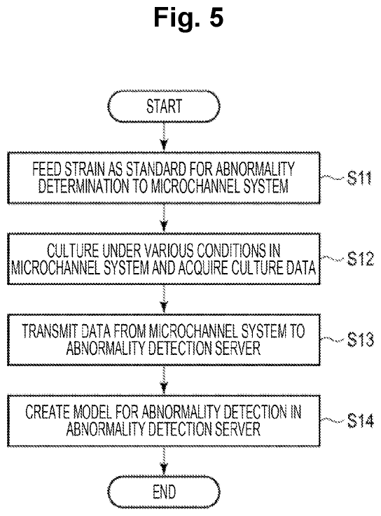

Next, creation of the abnormality detection model will be described. FIG. 5 is a flowchart illustrating an example of a procedure of creating an abnormality detection model.

First, a culture solution containing a culture strain which is a culture target strain of microalgae as a standard for abnormality determination from the mass culturing device 100 is fed to the microchannel system 200 (S11).

In the microchannel system 200, the culture strain contained in the culture solution fed in S11 is cultured by the channel chip 204 under various culture conditions. The control unit 222 of the control server 209 controls a large number of measurements by the measurement device 205 with respect to a cultured strain (sometimes referred to as a culture result) being cultured by the channel chip 204, and controls settings of culture conditions by the environment control device 206.

In the acquisition unit 223 of the control server 209, the measurement device 205 acquires a measurement result by the microscope system.

The data generation unit 224 of the control server 209 calculates the specific growth rate of the sample of the microalgae culture strain many times on the basis of the acquired measurement result. Then, the data generation unit 224 generates culture data including a large number of calculation results according to the normal distribution of the specific growth rate for each of a plurality of kinds of culture conditions controlled by the environment control device 206 or the like, and culture data including the specific growth rate calculated by the data generation unit 224 (S12).

This culture data can include (1) the culture result measurement date and time when the culture result of the trace sample is measured by the measurement device 205, (2) the sample No. for identifying the trace sample, (3) the sample acquisition date and time when the trace sample is acquired by the automatic pull liquid feeding device 203, and (4) the culture result No. for identifying the culture result associated with (1) to (3). The specific growth rate may be calculated by the control server 209 on the basis of a measurement result by the microscope system of the measurement device 205.

The communication unit 225 of the control server 209 of the microchannel system 200 transmits the culture data for each of the plurality of kinds of culture conditions acquired in S12 to the abnormality detection server 300 (S13).

The acquisition unit 302 of the abnormality detection server 300 acquires the culture data for each of the plurality of kinds of culture conditions transmitted in S13, and stores the culture data in the culture condition DB 301a of the storage device 301. The model creation unit 303 of the abnormality detection server 300 selects culture data related to one of a plurality of kinds of culture conditions from the stored culture data. Then, the model creation unit 303 obtains the average and variance of individual specific growth rates in the culture data under the selected condition, for example, on the basis of the T2 method of Hotelling, and calculates the degree of abnormality of the specific growth rate on the basis of the average and variance.

Then, the model creation unit 303 calculates an abnormality degree threshold which is a threshold at which the degree of abnormality becomes an outlier, and performs this calculation for each of the other culture conditions to create an abnormality detection model in which the abnormality degree threshold for each culture condition is defined, and stores this abnormality detection model in the abnormality detection model DB 301b of the storage device 301 (S14).

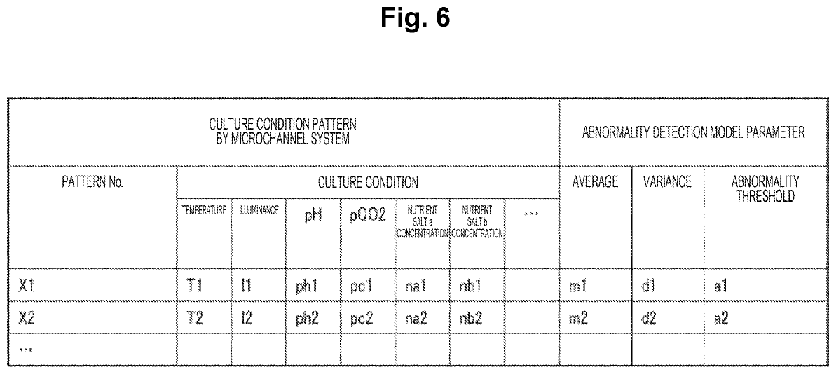

FIG. 6 is a table illustrating an example of the abnormality detection model.

As illustrated in FIG. 6, in the abnormality detection model, abnormality detection model parameters for each of a plurality of culture condition patterns by the microchannel system 200 are defined.

The culture condition pattern includes a pattern No. which is an identification number of each individual culture condition pattern and the content of the culture condition. The culture conditions include a temperature related to the channel chip 204 of the microchannel system 200, illuminance, ph and pCO₂ of a trace sample flowing through the channel of the channel chip 204, a plurality of kinds of nutrient salt concentrations, and the like. Note that the “nutrient salt a concentration” and the “nutrient salt b concentration” illustrated in FIG. 6 mean the first and second kinds of nutrient salt concentrations.

The above abnormality detection model parameters include the average, variance, and abnormality degree threshold of the specific growth rate.

For example, the first line of the table illustrated in FIG. 6 indicates that the abnormality detection model having an abnormality degree threshold “a1” under culture conditions including a temperature “T1” and illuminance “11” related to the culture condition pattern No. “X1” is created, and the second line of the table illustrated in FIG. 6 indicates that the abnormality detection model having an abnormality degree threshold “a2” under culture conditions including a temperature “T2” and illuminance “12” related to the culture condition pattern No. “X2” is created.

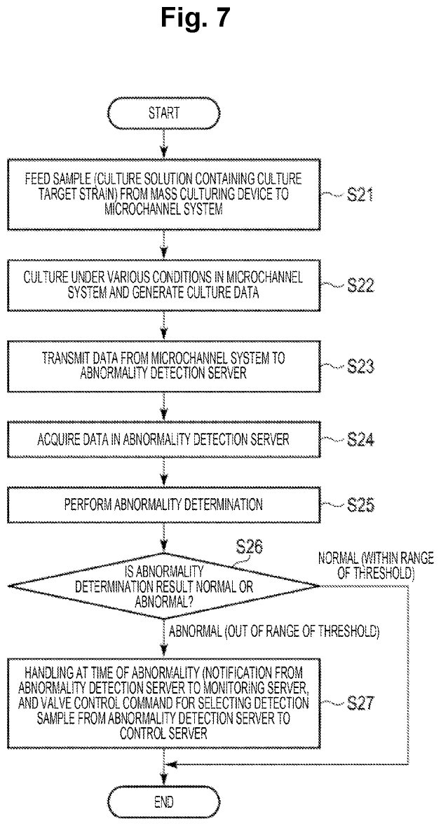

FIG. 7 is a flowchart illustrating an example of a procedure of mass culturing and abnormality detection.

Here, it is assumed that mass culturing by the mass culturing device 100 is started and continued, and the abnormality detection model is already created by the abnormality detection server 300.

First, a sample, that is, a culture solution containing a culture target strain being mass-cultured is fed from the mass culturing device 100 to the reservoir 202a of the microchannel system 200 (S21).

In the microchannel system 200, the sample fed in S21 is cultured by the channel chip 204 under various culture conditions, and the control unit 222 of the control server 209 controls the measurement by the measurement device 205 and controls the culture conditions via the environment control device 206 or the like. The measurement device 205 calculates the specific growth rate of the sample on the basis of the measurement result by the microscope system.

The acquisition unit 223 of the control server 209 acquires culture data including the specific growth rate calculated by the measurement device 205 for each of a plurality of kinds of culture conditions set by the environment control device 206 (S22).

The communication unit 225 of the control server 209 transmits the culture data acquired in S22 to the abnormality detection server 300 (S23).

The acquisition unit 302 of the abnormality detection server 300 acquires the culture data transmitted in S23 (S24).

The abnormality determination unit 304 of the abnormality detection server 300 selects culture data related to one of a plurality of kinds of culture conditions in the culture data acquired in S24. Then, the abnormality determination unit 304 calculates the degree of abnormality of each specific growth rate indicated by the selected culture data. The abnormality determination unit 304 reads an abnormality degree threshold related to the same culture condition as the selected culture condition in the abnormality detection model stored in the abnormality detection model DB 301b of the storage device 301. The abnormality determination unit 304 performs abnormality determination, which is determination of the presence or absence of abnormality during the mass culturing, for each of the plurality of culture conditions by comparing the read abnormality degree threshold with the calculated degree of abnormality (S25).

In the determination in S25, when the degree of abnormality based on the specific growth rate indicated by the culture data, which is the degree of abnormality compared under a certain culture condition, exceeds the abnormality degree threshold, the abnormality determination unit 304 determines that the result of the abnormality determination is “abnormal”, that is, that an abnormality has occurred during the mass culturing under this culture condition (“abnormal” in S26).

When this determination is made, the response at the time of abnormality is performed (S27). Examples of the response at the time of abnormality include abnormality occurrence notification to the monitoring server 400 by the notification unit 305 of the abnormality detection server 300, and output of a valve control command for feeding a sample to the reservoir 208b, assuming that the sample fed to the channel chip 204 at the time of determining occurrence of abnormality is a sample in which abnormality is observed during the mass culturing, from the abnormality detection server 300 to the control server 209 of the microchannel system 200.

On the other hand, when the degree of abnormality does not exceed the abnormality degree threshold, the abnormality determination unit 304 determines that the result of the abnormality determination is “normal”, that is, no abnormality has occurred during the mass culturing of the cultured strain (“normal” in S26). At the time of this determination or after the response to the abnormality, the abnormality determination unit 304 stores a series of abnormality determination results in the storage device 301, for example, and the processing ends.

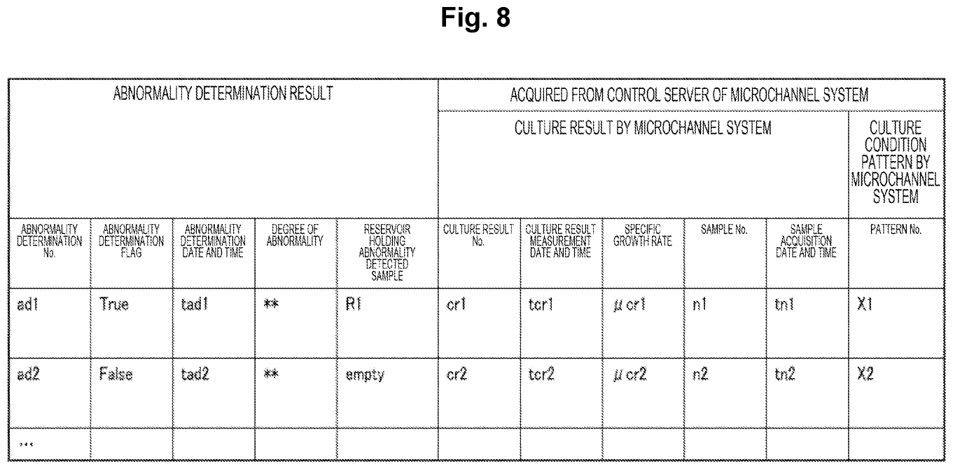

FIG. 8 is a table illustrating an example of an abnormality determination result in a tabular form.

In the example illustrated in FIG. 8, the abnormality determination result is generated for each of the culture condition patterns in the culture data acquired in S24 from the control server 209 of the microchannel system 200.

In addition, the abnormality determination result includes (1) an abnormality determination flag indicating the presence or absence of abnormality, (2) a determination date and time of the presence or absence of abnormality, (3) a degree of abnormality of the specific growth rate calculated in the determination of the presence or absence of abnormality, (4) reservoir information specifying a reservoir in which the sample fed to the channel chip 204 is held when it is determined that there is abnormality, and (5) an abnormality determination No. identifying an abnormality determination result associated with the above (1) to (4).

In addition, in the example illustrated in FIG. 8, culture results in the microchannel system 200 under each culture condition are illustrated, and the culture results include the culture result No., the culture result measurement date and time, the specific growth rate, the sample No., and the sample acquisition date and time.

For example, the abnormality determination result of an abnormality determination No. “ad1” in the first row of the table illustrated in FIG. 8 indicates that under the culture condition pattern “X1”, the abnormality determination flag at an abnormality determination date and time “tad1” is “True”, that is, an abnormality during the mass culturing is observed.

In addition, the abnormality determination result of an abnormality determination No. “ad2” in the second row of the table illustrated in FIG. 8 indicates that under the culture condition pattern “X2”, the abnormality determination flag at an abnormality determination date and time “tad2” is “False”, that is, no abnormality during the mass culturing is observed.

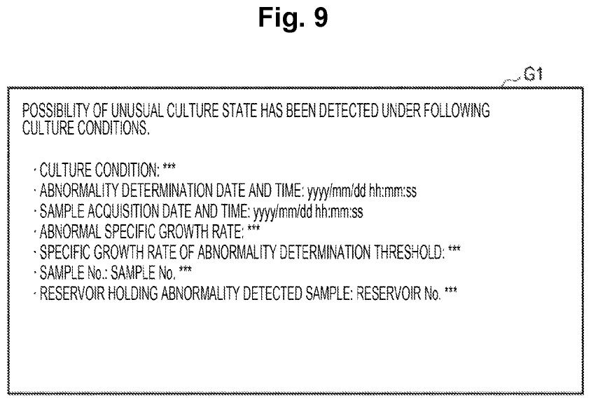

FIG. 9 is a diagram illustrating an example of a notification screen for abnormality detection.

A screen G1 illustrated in FIG. 9 is a screen displayed on an external display device, not illustrated, by the display processing unit 306 of the abnormality detection server 300 when the determination result by the abnormality detection server 300 is a result indicating that an abnormality has been observed during the mass culturing.

This screen displays culture conditions, abnormality determination date and time, sample acquisition date and time, specific growth rate at the time of abnormality determination, a threshold of a degree of abnormality of the specific growth rate (specific growth rate of abnormality determination threshold), a sample No., and a reservoir in which the sample is held at the time of abnormality detection, together with a message “the possibility of an unusual culture state has been detected under the following culture conditions”.

In the embodiment described above, a trace sample containing a culture target strain and a culture solution during the mass culturing in a mass culturing device is fed to a microchannel system, culture is performed under various culture conditions in the microchannel system, culture data is acquired, and an abnormality during the mass culturing is detected using the data.

Since this microchannel system can culture a small-scale sample, the cost of data acquisition can be made relatively small.

In addition, since culture data can be acquired by culturing a sample in the microchannel system under a condition different from the culture condition by the mass culturing device, an abnormality during mass culturing can be detected at an early stage.

Although there is a possibility that the culture efficiency is lowered due to the occurrence of contamination or genetic mutation in a strain exhibiting excellent culture efficiency under specific culture conditions during culture, in the present embodiment, culture data can be acquired by culturing a sample under various culture conditions in the microchannel system, and thus the occurrence of contamination or genetic mutation can be appropriately detected.

In addition, in the mass culturing of microalgae, outdoor raceway pounds may be used, but in this method, contamination organisms are likely to be mixed, and it is difficult to predict the timing of mixing. As an example of contamination organisms, there is a ciliate that is in a dormant state at the time of contamination but wakes up at a specific temperature condition to affect culture. In the present embodiment, even in such a case, a change in culture efficiency due to predation by a waking up nematode can be detected because the contamination organism is placed in a specific temperature condition in the microchannel system before actually waking up from the dormant state in the mass culturing device.

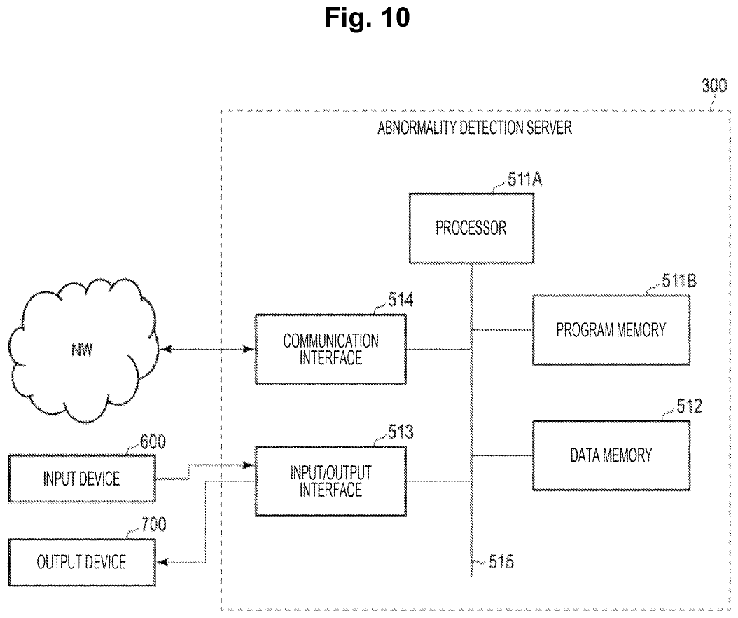

FIG. 10 is a block diagram illustrating an example of a hardware configuration of the abnormality detection server 300 according to an embodiment of the present invention.

In the example illustrated in FIG. 10, the abnormality detection server 300 according to the aforementioned embodiment is, for example, a server computer or a personal computer and includes a hardware processor 511A such as a CPU. To this hardware processor 511A, a program memory 511B, a data memory 512, an input/output interface 513, and a communication interface 514 are connected via a bus 515. Hereinafter, the abnormality detection server 300 will be described as an example, but the same applies to the control server 209, the monitoring server 400, and the like in the microchannel system 200.

The communication interface 514 includes, for example, one or more wireless communication interface units and transmits and receives information to and from a communication network NW. An example of a wireless interface used herein includes an interface that employs a low-power wireless data communication standard such as a wireless Local Area Network (LAN).

To the input/output interface 513, an input device 600 and an output device 700 attached to the abnormality detection server 300 and used by a user or the like are connected.

The input/output interface 513 captures operation data inputted by a user or the like through the input device 600 such as a keyboard, a touch panel, a touchpad, and a mouse, outputs the data to the output device 700 including a display device using liquid crystals or organic Electro Luminescence (EL), and allows the output device 700 to display the output data. Note that, as the input device 600 and the output device 700, a device built in the abnormality detection server 300 may be used, and an input device and an output device of another information terminal that can communicate with the abnormality detection server 300 via the network NW may be used.

The program memory 511B is a non-transitory tangible storage medium in which, for example, a non-volatile memory enabling writing and reading at any time such as a Hard Disk Drive (HDD) and a Solid State Drive (SSD) and a non-volatile memory such as a Read Only Memory (ROM) are combined, and the program memory 511B stores programs required for executing various types of control processing according to an embodiment.

The data memory 512 is a tangible storage medium in which, for example, the aforementioned non-volatile memory and a volatile memory such as a Random Access Memory (RAM) are combined and is used for storing various types of data acquired and generated during the course of various types of processing.

The abnormality detection server 300 according to an embodiment of the present invention can be configured as a data processing device that includes a processing functional unit using software.

The storage device 301 used as a working memory or the like by the abnormality detection server 300 can be configured by using the data memory 512 illustrated in FIG. 10. However, these storage areas are not necessarily included in the abnormality detection server 300 and may be areas in, for example, an external storage medium such as a Universal Serial Bus (USB) memory or a storage device such as a database server in the cloud.

The aforementioned processing functional unit is achieved by causing the hardware processor 511A to read and execute the programs stored in the program memory 511B.

Note that the processing functional unit may be achieved in other various forms including an integrated circuit such as an Application Specific Integrated Circuit (ASIC) and a Field-Programmable Gate Array (FPGA).

Moreover, the methods described in each embodiment can be stored in a recording medium such as a magnetic disk (Floppy (registered trademark) disk, hard disk, and the like), an optical disc (CD-ROM, DVD, MO, and the like), or a semiconductor memory (ROM, RAM, flash memory, and the like) as a program (software means) that can be executed by a computer, and can be distributed by being transmitted through a communication medium. Note that the programs stored in the medium also include a setting program for configuring, in the computing machine, software means (including not only an execution program but also a table and a data structure) to be executed by the computing machine. The computing machine that implements the present device executes the above-described processing by reading the programs recorded in the recording medium, constructing the software means by the setting program as needed, and controlling operation by the software means. Note that the recording medium in the present specification is not limited to a recording medium for distribution, and includes a storage medium such as a magnetic disk or a semiconductor memory provided in equipment connected inside a computer or via a network.

Note that the present invention is not limited to the above-described embodiment, and various modifications can be made at the implementation stage without departing from the gist of the invention. In addition, embodiments may be implemented in appropriate combination, and in this case, a combined effect can be obtained. In addition, the embodiment described above include various aspects of the invention, and the various aspects of the invention can be extracted by combinations selected from a plurality of disclosed constituent elements. For example, in a case where the problems can be solved and the advantageous effects can be obtained even if some constituent elements are deleted from all the constituent elements described in the embodiment, a configuration from which the constituent elements are deleted can be extracted as an invention.

### REFERENCE SIGNS LIST

100 Mass culturing device
200 Microchannel system
201 Automatic push liquid feeding device
202a, 202b, 202c, 202d, 208a, 208b Reservoir
203 Automatic pull liquid feeding device
204 Channel chip
205 Measurement device
206 Environmental control device
207 Valve control device
209 Control server
300 Abnormality detection server
400 Monitoring server

1. An abnormality detection device comprising:
a memory device; and
at least one hardware processor configured to:
measure a culture state of a sample of a culture target that is a culture target during mass culturing by a mass culturing device and that is fed to a microchannel chip and cultured under a set culture condition; and
detect an abnormality in the culture state of the sample of the culture target by comparing an abnormality detection model in which a criterion for determining an abnormality in the culture state is defined for each of a plurality of culture conditions, and the culture state measured for the sample and the set culture condition, wherein the criterion for determining the abnormality indicates that the culture state of the sample of the culture target is different from a normal culture state.

2. The abnormality detection device according to claim 1, wherein
the at least one hardware processor is configured to
measure the culture state of the sample of the culture target which is fed to the microchannel chip and cultured under each of a plurality of kinds of culture conditions, and
create, on a basis of the measured culture state, the abnormality detection model in which the criterion for determining the abnormality in the culture state of the sample of the culture target is defined for each of the plurality of kinds of culture conditions.

3. The abnormality detection device according to claim 1, wherein
the culture conditions comprise:
at least one of a temperature related to a channel of the sample in the microchannel chip, an illuminance with respect to the channel, a hydrogen ion concentration index of the sample, a carbon dioxide partial pressure of the sample, or a nutrient salt concentration of the sample.

4. The abnormality detection device according to claim 1, wherein
a culture state of the sample comprises:
a specific growth rate, a specific substrate consumption rate, a product specific production rate, or a specific oxygen consumption rate of a sample of a culture target that is fed to the microchannel chip and cultured.

5. An abnormality detection method performed by an abnormality detection device, the abnormality detection method comprising:
measuring, by at least one hardware processor of the abnormality detection device, a culture state of a sample of a culture target that is a culture target during mass culturing by a mass culturing device and that is fed to a microchannel chip and cultured under a set culture condition; and
detecting, by the at least one hardware processor of the abnormality detection device, an abnormality in the culture state of the sample of the culture target by comparing an abnormality detection model in which a criterion for determining an abnormality in the culture state is defined for each of a plurality of culture conditions, and the culture state measured for the sample and the set culture condition, wherein the criterion for determining the abnormality in the culture state indicates that the culture state of the sample of the culture target is different from a normal culture state.

6. The abnormality detection method according to claim 5, further comprising:
measuring the culture state of the sample of the culture target which is fed to the microchannel chip and cultured under each of a plurality of kinds of culture conditions, and
creating, on a basis of the measured culture state, the abnormality detection model in which the criterion for determining the abnormality in the culture state of the sample of the culture target is defined for each of the plurality of kinds of culture conditions.

7. The abnormality detection method according to claim 5, wherein
the culture conditions comprise:
at least one of a temperature related to a channel of the sample in the microchannel chip, an illuminance with respect to the channel, a hydrogen ion concentration index of the sample, a carbon dioxide partial pressure of the sample, or a nutrient salt concentration of the sample.

8. A non-transitory computer-readable medium storing instructions of an abnormality detection processing program, wherein execution of the instructions by at least one hardware processor causes the at least one hardware to perform operations comprising:
measuring a culture state of a sample of a culture target that is a culture target during mass culturing by a mass culturing device and that is fed to a microchannel chip and cultured under a set culture condition; and
detecting an abnormality in the culture state of the sample of the culture target by comparing an abnormality detection model in which a criterion for determining an abnormality in the culture state is defined for each of a plurality of culture conditions, and the culture state measured for the sample and the set culture condition, wherein the criterion for determining the abnormality in the culture state indicates that the culture state of the sample of the culture target is different from a normal culture state.
